(19) United States
(12) Patent Application Publication
(10) Pub. No.: US 2010/0003332 A1
(43) Pub. Date: Jan. 7, 2010

(54) Process For Preparing Powder Comprising Nanoparticles of Sparingly Soluble Drug

(75) Inventors: Joon Ho Bae, Yongin-si (KR); Jong Hwi Lee, Seoul (KR); Hyeok Lee, Seongnam-si (KR); Jung Ju Kim, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/375,232

(22) PCT Filed: Jul. 26, 2007
(86) PCT No.: PCT/KR07/03599

§ 371 Date: Jan. 27, 2009

(30) Foreign Application Priority Data
Jul. 27, 2006 (KR) 10-2006-0070556

(51) Int. Cl. A61K 9/14; A61K 31/19; A61K 31/4353; A61K 31/47; A61K 31/216; A61K 31/496; A61P 43/00

(52) U.S. Cl. 424/489; 514/569; 514/291; 514/312; 514/543; 514/254.07

(57) ABSTRACT

A powder comprising nanoparticles of a sparingly water-soluble drug prepared in accordance with the present invention exhibits enhanced bioavailability without generating adverse side effects caused by impurities, while the nano-particle size of the drug remains unchanged when administered. Accordingly, the powder can be useful for the development of a formulation of a sparingly water-soluble drug for oral and parenteral administration.

### FIELD OF THE INVENTION

The present invention relates to a process for preparing a powder composition comprising nanoparticles of a sparingly water-soluble drug, which exhibits enhanced bioavailability and particle size stability of the drug, when dispersed in an aqueous medium.

### BACKGROUND OF THE INVENTION

Bioavailability is a pharmacokinetic parameter defined by the amount of a drug absorbed based on the amount administered, which is used to determine the effectiveness of an administered pharmaceutical active ingredient or a formulation comprising same. The characteristics of a pharmaceutical active ingredient, e.g., water-solubility, crystal forms and particle size of the active ingredient, can affect the bioavailability of the active ingredient or a composition comprising same. For example, the sparingly water-soluble drug itself is not absorbed in the gastrointestinal tract, leading to poor bioavailability. Moreover, the use of various dispersion agents or surfactants to alleviate the difficulty of formulating a sparingly water-soluble drug for parenteral administration such as injections leads to undesired side effects.

Accordingly, there have been numerous attempts to develop improved methods for preparing a sparingly water-soluble drug in the forms of: a specific crystal form (Korean Patent Publication No. 1999-15201); a clathrate (U.S. Pat. No. 6,407,079); a solid dispersion (International Patent Publication WO98/046268); a microemulsion (International Patent Publication WO93/020833); micelles using an amphiphilic copolymer; and nanoparticles (Korean Patent Publication No. 1999-69033).

However, compositions prepared by the above-mentioned methods may bring about undesired side effects due to the presence of a solvent, dissolution adjuvant, or surfactant used to improve the solubility and bioavailability of the sparingly water-soluble drug. Also, the drug stability in such powder compositions tends to be poor under typical storage conditions. Further, the cost of preparing such composition is high due to the fact that such methods require the use of complex processes and expensive ingredients.

There have also been attempts to improve the water-solubility and bioavailability of a sparingly water-soluble drug by preparing a powder composition thereof. For example, U.S. Pat. No. 5,145,684 and Korean Patent Publication No. 1992-14468 disclose a method of preparing a powder composition comprising an active ingredient having an average particle size less than 400 nm, by dispersing the sparingly water-soluble drug in an aqueous medium, and wet grinding, e.g., milling, in the presence of a surface modifier or adding a surface modifier after grinding. Korean Patent Publication No. 2003-67713 disclose a method of preparing nanoparticles or powders of a drug comprising the steps of: first dissolving an active ingredient in a water-miscible organic solvent, and then, adding a solvent which does not dissolve the drug such as water thereto, to obtain a presuspension of the drug having an effective average particle size of 2 μm, which is similar to the method disclosed in U.S. Pat. No. 5,145,684.

However, the compositions prepared by above-mentioned methods are all of the form of aqueous dispersions, which requires an additional spray drying or freeze drying step for obtaining a solid form of the drug. Moreover, the particles of the sparingly water-soluble drug prepared by the above methods tend to agglomerate when redispersed in an aqueous medium after drying.

### SUMMARY OF THE INVENTION

Accordingly, it is an object of the present invention to provide a process for preparing a powder composition comprising nanoparticles of a sparingly water-soluble drug, which exhibits enhanced bioavailability and particle size stability of the drug when dispersed in an aqueous medium.

It is another object of present invention to provide a powder composition prepared in accordance with the above-mentioned process.

It is still another object to provide a pharmaceutical composition comprising such powder composition.

In accordance with one aspect of the present invention, there is provided a method for preparing a powder composition comprising nanoparticles of a sparingly water-soluble drug comprising:

1) dispersing particles of the sparingly water-soluble drug, a surface stabilizer and a dispersion agent in a saturated aqueous solution of the dispersion agent to obtain a dispersion;

2) mixing and grinding the dispersion obtained in step 1) to obtain a homogenized dispersion; and

3) centrifuging or high-pressure filtering the homogenized dispersion obtained in step 2) to isolate a solid, and drying the solid to obtain a powder.

In accordance with another aspect of the present invention, there is provided a powder composition comprising the particles of the sparingly water-soluble drug prepared by the inventive method, which shows a particle size distribution of 10 to 1000 nm for 10 to 90% of the drug particles determined based on a particle size normal distribution curve obtained for the powder and an average particle size of 10 to 400 nm in an aqueous medium.

In accordance with still another aspect of the present invention, there is provided a pharmaceutical composition comprising the powder composition together with a pharmaceutically acceptable carrier.

### BRIEF DESCRIPTION OF DRAWINGS

The above and other objects and features of the present invention will become apparent from the following description of the invention, when taken in conjunction with the accompanying drawings:

FIG. 1: a graph showing the particle size distribution of the active ingredient particles when a slurry mixture and a drying powder obtained after wet-grinding according to the present invention was redispersed in distilled water, respectively;

FIG. 2: a graph showing the particle size distribution of the active ingredient particles according to the redispersion method of the powder; and

FIG. 3: a graph showing the concentration result versus time measured after suspending cilostazol-containing powder and unprocessing cilostazol material in distilled water and administrating the suspensions to rats, respectively.

### DETAILED DESCRIPTION OF THE INVENTION

The method of preparing the powder composition in accordance with the present invention is characterized by comprising the steps of mixing nanoparticles of a sparingly water-soluble drug, a surface stabilizer and a dispersion agent, and then, drying the mixture to obtain the inventive powder.

The method of preparing the powder comprising the sparingly water-soluble drug according to the present invention is described in detail as follows:

### Step 1: Preparation of a Dispersion

In the step 1) of the present invention, a dispersion is prepared by adding particles of the active sparingly water-soluble drug to a solution containing a surface stabilizer and a dispersion agent to obtain a dispersion, wherein the concentration of the dispersion agent in said solution is saturation concentration.

1-1) Active Ingredient

The sparingly water-soluble drug used in the present invention is an organic compound first dispersed in an aqueous medium. The dispersion may contain an alcohol, and “sparingly water-soluble drug” as used herein means a drug having a solubility of less than 10 mg/ml, preferably less than 1 mg/ml in an aqueous medium at room temperature.

Representative examples of the sparingly water-soluble drug include non-steroidal anti-inflammatory drugs including acetaminophen, acetylsalicylic acid, ibuprofen, penbuprofen, fenoprofen, flubiprofen, indomethacin, naproxen, etorolac, ketoprofen, dexibuprofen, piroxicam and aceclofenac; immunosuppressants or therapeutic agents for atopic dermatitis including cyclosporine, tacrolimus, rapamycin, mycophenylate and pimecrolimus; calcium channel blockers including nifedipine, nimodipine, nitrendipine, nilvadipine, felodipine, amlodipine and isradipine; angiotensin II receptor antagonists including valsartan, eprosartan, irebesartan, candersartan, telmisartan, olmesartan and losartan; therapeutic agents for hyperlipidemia inhibiting cholesterol synthesis including atorvastatin, lovastatin, simvastatin, fluvastatin, rosuvastatin and pravastatin; therapeutic agents for hyperlipidemia promoting cholesterol metabolism and secretion including gemfibrozil, fenofibrate, etofibrate and bezafibrate; therapeutic agents for diabetes including pioglitazone, rosiglitazone and metformin; lipase inhibitors including orlistat; antifungal drugs including itraconazole, amphotericin B, terbinafine, nystatin, griseofulvin, fluconazole and ketoconazole; liver protectors including biphenyl dimethyl dicarboxylate, silymarin and ursodesoxycholic acid; therapeutic agents for digestive tract disease including sopharcone, omeprazole, pantoprazole, famotidine, itopride and mesalazine; platelet aggregation inhibitors including cilostazol and clopidogrel; therapeutic agents for osteoporosis including raloxifene; antiviral agents including acyclovir, famciclovir, lamivudine and oseltamivir; antibiotics including clarithromycin, ciprofloxacin and cefuroxime; antiasthmatic drugs and antihistamines including pranlukast, budesonide and fexofenadine; hormones including testosteron, prednisolone, estrogen, cortisone, hydrocortisone and dexamethasone; antitumor agents including paclitaxel, docetaxel, paclitaxel derivatives, doxorubicin, adriamycin, daunomycin, campothecin, etoposide, teniposide and busulfan; and salts, pharmaceutical derivatives, and a mixture thereof, and preferably naproxen, tacrolimus, valsartan, simvastatin, fenofibrate, itraconazole, biphenyl dimethyl dicarboxylate, silymarin, sopharcone, pantoprazole, cilostazol, and salts, pharmaceutical derivatives and a mixture thereof.

The particle size of the sparingly water-soluble drug used in step 1) of the present invention does not limit the scope of the present invention, but it is preferred that the step of treating the sparingly water-soluble drug is carried out using a conventional milling method such as airjet or fragmentation milling to form particles having an average particle size of less than 100 μm, before conducting step 1).

The particles of sparingly water-soluble drug content of said dispersion may be in the range of 0.1 to 60% by weight, preferably 4 to 40% by weight in the saturated aqueous solution containing the dispersion agent.

1-2) Surface Stabilizer

The surface stabilizer used in the present invention can be any of pharmaceutically acceptable organic or inorganic compounds which do not chemically react with the active ingredient or the dispersion agent.

Representative examples of the surface stabilizer include sodium dodecylsulfate (SDS), sodium lauryl sulfate (SLS), sodium dioctyl sulfosuccinate, lecithin, phospholipid, polyoxyethylene sorbitan fatty acid ester (e.g. Tween®), potassium sorbate, poloxamer, prophylene glycol, methyl cellulose, ethyl cellulose, hydroxymethyl cellulose, hydroxyethyl cellulose, hydroxypropyl cellulose, hydroxypropyl methylcellulose, carboxymethyl cellulose, benzethonium chloride, benzalkonium chloride, sorbic acid, benzoic acid, sodium benzoate, propylparaben, methylparaben, polyvinylalcohol, polyvinylpyrrolidone, alginic acid, sodium alginate and a mixture thereof, and preferably, hydroxypropyl cellulose, poloxamer, polyvinylpyrrolidone and a mixture thereof.

In the present invention, the surface stabilizer can be used in an amount ranging from 0.0001 to 90% by weight, preferably 0.01 to 50% by weight, more preferably 0.1 to 20% by weight based on the weight of the sparingly water-soluble drug.

1-3) Dispersion Agent

The dispersion agent used in the present invention is of the form of particles which are added to its saturated aqueous solution so as to increase the apparent viscosity of the dispersing solution, which allows the formation of small and homogenous particles of the sparingly soluble drug containing particles of the dispersion agent during the milling process. In order that the dispersion agent exists in the form of particles, it should be present in at least saturated concentration in the dispersion.

Representative examples of such dispersion agent include monosaccharides, disaccharides and trisaccharides such as lactose, sucrose, raffinose, mannitol, trehalose, sorbitol, xylitol, glycerol, dextrose and fructose, and a mixture thereof.

The dispersion agent may be used in an amount ranging from 0.1 to 200% by weight, preferably 20 to 180% by weight, more preferably 60 to 140% by weight based on the weight of the sparingly water-soluble drug.

1-4) Solvent

The solvent used in the present invention may be water, an aqueous or buffer solution, and it may contain an alcohol in an amount of less than 50% by weight depending on the properties of the active ingredient. The alcohol which may be employed in the present invention includes methyl alcohol, ethyl alcohol, propyl alcohol and a mixture thereof.

### Step 2: Homogenization of the Dispersion

In step 2) of the present invention, the dispersion obtained in step 1) is mixed and ground to homogenize the dispersion while reducing the particle size of the sparingly water-soluble drug. The mixing and grinding process may be conducted by a wet grinding process using a dispersion mill including a ball mill, an oscillating mill and a bead mill; an ultrasonic irradiation process; or a shearing force grinding process. The process temperature and the process time can be adjusted according to the kind of the active ingredient, and the process can be carried out at room temperature for period of several minutes to several days. The homogenized dispersion obtained in step 2) has an apparent viscosity ranging from 1 to 100,000 centipoises, preferably 10 to 50,000 centipoises, more preferably 500 to 10,000 centipoises. As the process time of step 2) is longer, the particle size of the active ingredient becomes smaller and more homogeneous.

### Step 3: Collection of Powder

For the purpose of higher production efficiency and cost cutting, the homogenized dispersion obtained in step 2) is centrifuged or high-pressure filtered to remove the solvent, followed by drying to collect the powder.

The centrifuging process can be conducted at a rate ranging from 500 to 200,000 rpm, preferably 1,500 to 80,000 rpm and at a temperature ranging from 0 to 50° C., preferably 1 to 30° C. for 5 to 400 minutes, preferably 30 to 300 minutes. The high-pressure filtering process can be conducted at a pressure ranging from 200 to 2,000 mmHg, preferably 500 to 1,000 mmHg and at a temperature ranging from 0 to 50° C., preferably 1 to 30° C. for 5 to 400 minutes, preferably 10 to 200 minutes. The drying process can be conducted by using any of the conventional drying methods such as freeze drying or spray drying.

The powder composition obtained in accordance with the present invention comprises a crystalline form of the sparingly water-soluble drug having a particle size distribution of 10 to 1000 nm for 10 to 90% (D10˜D90) of the drug particles determined based on a particle size normal distribution curve obtained for the powder. Further, it has an average particle size of 10 to 400 nm in an aqueous solution including a buffer. Further, the inventive powder can be easily redispersed in an aqueous medium through, e.g., simple mechanical stirring and ultrasonic irradiation. The redispersed particles of the sparingly water-soluble drug have more or less the same average particle size of the original powder form thereof.

The powder composition obtained in accordance with the present invention has a nanoparticle size and it comprises particles of the active ingredient together with the surface stabilizer and the dispersion agent particles homogeneously mixed therein, but it is not of a form wherein the surface stabilizer or the dispersion agent are absorbed on the surface of the active ingredient particles. The average particle size of the surface and the dispersion agent is on the nano- or micro-level.

Accordingly, the powder comprising the sparingly water-soluble drug obtained in accordance with the present invention exhibits enhanced bioavailability without generating adverse side effects caused by impurities, while the nano-particle size of the drug remains unchanged when administered. Accordingly, the powder can be useful for the development of a formulation of a sparingly water-soluble drug for oral and parenteral administration.

In addition, the present invention provides a pharmaceutical composition comprising the powder according to the present invention together with a pharmaceutically acceptable carrier. The pharmaceutical composition may be of a preparation form selected from the group consisting of granules, powders, syrups, liquids, suspensions, tablets, capsules, troches or pills for oral administration, and transdermal systems, lotions, ophthalmic ointments, ointments, plasters and pressure sensitive adhesives, cataplasmas, creams, pastes, suspensions, liquids, injections or suppository for parenteral administration.

The following Examples are given for the purpose of illustration only, and are not intended to limit the scope of the invention.

### Example 1

### Particle Size Variation of the Active Ingredient with the Kind of the Dispersion Agent Used

In order to observe the particle size variation of the active ingredient with the kind of the dispersion agent used, a number of powders were prepared using naproxen as the active ingredient and using various dispersion agents:

0.15 g of naproxen (TCI Chem) having an average particle size of 3 to 10 μm, 0.15 g of lactose and 0.03 g of hydroxypropy cellulose (HPC) were added to 3.4 ml of a saturated aqueous solution of lactose, and the mixture was wet ground using an oscillating mill at the room temperature for 30 minutes. The slurry mixture thus obtained was centrifuged at 4° C., 15,000 rpm for 30 minutes, and the residue in the bottom layer was vacuum dried for 24 hours to obtain a powder.

In the above process, the averaged particle size of naproxen particles was measured for 1) a test solution obtained by redispering 0.02 ml of the slurry mixture obtained after wet grinding in 10 ml of distilled water, and 2) an another test solution obtained by redispersing 0.01 g of the dried powder in 8 ml of distilled water, by using a laser scattering particle size analyzer (LA-910, Horiba). The redispersion was conducted by shaking the mixture with a hand.

As a result, it was observed that when the slurry mixture obtained after wet grinding was redispersed in distilled water, the average particle size of naproxen particles was about 133 nm, and when the dried powder was redispersed in distilled water, the average particle size of naproxen particles was about 164 nm. Therefore, the particle of the sparingly water-soluble drug in the powder obtained according to the present invention remained unchanged at nano-level when redispersed.

Additional powders were prepared by repeating the above-mentioned procedure except for using sucrose, mannitol, trehalose, sorbitol and xylitol, respectively, instead of lactose, and naproxen particle sizes were measured by the same procedure. The results are shown in Table 1.

TABLE 1

| | | | | Average |
|---|---|---|---|---|
| Active | Surface | Dispersion | Particle Size | particle |
| ingredient | stabilizer | agent | (D10~D90) (nm) | size (nm) |
| Naproxen | HPC | Lactose | 40.3~259.6 | 164.1 |
| | | Sucrose | 192.9~393.9 | 297.1 |
| | | Mannitol | 319.8~564.6 | 450 |
| | | Trehalose | 162.8~312.5 | 241.6 |
| | | Sorbitol | 280.4~532.7 | 413.6 |
| | | Xylitol | 291.3~675.9 | 637.9 |

As shown in Table 1, the particle sizes of the active ingredient remained at nano-level when redispersed in distilled water.

### Example 2

### Particle Size Variation of the Active Ingredient with the Molecular Weight of the Surface Stabilizer

In order to examine how the particle size variation of the active ingredient is affected to the molecular weight of the surface stabilizer, powders were prepared by repeating the procedure of Example 1 except for using polyvinylpyrrolidones having molecular weights of 10,000, 29,000, 55,000 and 130,000, respectively, instead of hydroxypropyl cellulose as the surface stabilizer.

The average particle sizes of the active ingredient were measured by the same method as in Example 1, and the results are shown in Table 2.

TABLE 2

| | | | | Average |
|---|---|---|---|---|
| Active | Dispersion | Surface | Particle Size | particle |
| Ingredient | agent | stabilizer | (D10~D90) (nm) | size (nm) |
| Naproxen | Lactose | PVP 10,000 | 868.3~507.1 | 16,800 |
| | | PVP 29,000 | 95.9~208.7 | 156.9 |
| | | PVP 55,000 | 120.8~250.3 | 192.4 |
| | | PVP 130,000 | 150.6~417.5 | 275.8 |

As shown in Table 2, when inventive powders were redispersed in distilled water, the particle size of the active ingredient remained at nano-level.

### Example 3

### Particle Size Variation of the Active Ingredient with the Amount of the Dispersion Agent

In order to examine how the particle size variation of the active ingredient is affected to the amount of the dispersion agent, powders were prepared by repeating the procedure of Example 1 except for using lactoses in amounts of 180, 140, 100, 60 and 20% by weight based on the weight of naproxen.

The average particle sizes of the active ingredient were measured by the same method as in Example 1, and the results are shown in Table 3.

TABLE 3

| | | Amount of lactose | Particle | |
|---|---|---|---|---|
| | | (% by weight based | Size | Average |
| Active | Surface | on the weight of the | (D10~D90) | particle |
| ingredient | stabilizer | active ingredient) | (nm) | size (nm) |
| Naproxen | HPC | 200% by weight * | — | — |
| | | 180% by weight | 108~310 | 208 |
| | | 140% by weight | 109~316 | 391 |
| | | 100% by weight | 40~260 | 164 |
| | | 60% by weight | 339~629 | 500 |
| | | 20% by weight | 154~4,292 | 3,025 |

* Nanoparticles are not formed due to the significant increase of the viscosity.

As shown in Table 3, As the amount of lactose is smaller, the average particle size of the active ingredient becomes larger, and when the amount of lactose is reduced to 20% by weight based on the weight of the active ingredient, the particle size of the active ingredient does not remained at nano-level.

### Example 4

### Particle Size Variation the Active Ingredient with the Kind of the Surface Stabilizer and Condition of the Redispersion

In order to observe the particle size variation of the active ingredient with the kind of the surface stabilizer and condition of the redispersion, powders were prepared as follows:

0.225 g of tacrolimus, 0.045 g of the surface stabilizer listed in Table 4 and 0.225 g of lactose were added to 5.1 ml of the saturated aqueous solution of lactose and the mixture was wet ground using a rotary mill at 4° C., 3,000 rpm for 30 minutes. The slurry mixture thus obtained was centrifuged at 4° C., 15,000 rpm for 30 minutes, and then the residue in the bottom layer was vacuum dried for 24 hours to obtain powders.

0.01 g of each of the powders thus obtained was redispersed using an ultrasonic irradiation (frequency: 39 kHz). The particle of the tacrolimus was measured by using a laser scattering particle size analyzer (LA0910, Horiba) for each of the test solution with and without the ultrasonic irradiation, and the results are shown in Table 4.

TABLE 4

| | Particle size according to the | | |
|---|---|---|---|
| | ultrasonic irradiation time | | |
| Surface stabilizer | | 0 minute | 1 minute |

| | | | |
|---|---|---|---|
| Hydroxyproply cellulose | 4,980 | nm | 220 nm |
| Poloxamer 407 | 510 | nm | 420 nm |
| Polyvinylpyrrolidone | 1,561 | nm | 610 nm |

As shown in Table 4, the particle size of the active ingredient was varied with the kind the surface stabilizer, and the average particle size of the active ingredient remained at nano-level under the ultrasonic irradiation for a short period of time.

The ultrasonic irradiation in Examples is not an operation or a process for processing and grinding the active ingredient to the nanoparticles. A degree of the redispersion may be different according to the ingredients of the surface stabilizer, but the particle size of the active ingredient remained at nano-level.

### Example 5

### Particle Size Variation of the Active Ingredient with the Content Ratio of the Dispersion Agent and the Surface Stabilizer

In order to observe particle size variation of the active ingredient with the content ratio of the dispersion agent and the surface stabilizer, powders were prepared as follows:

0.45 g of cilostazol (Dongwoo Co., Ltd.) and 0.15 g of hydroxypropyl cellulose were added to 5.1 ml of the saturated aqueous solution of lactose. Lactose and sodium lauryl sulfate were added thereto in the weight ratios of 4:1, 1:1 and 1:4(360 g: 90 g, 255 g: 255 g, 90 g: 360 g) and the mixture was wet ground using a rotary mill at 3,000 rpm for 30 minutes. The slurry mixture thus obtained was high-pressure filtered (pressure: 640 mmHg) using a sound pressure and freeze dried for 1 day to obtain powders.

In the above process, the average particle size of cilostazol was measured for 1) a test solution obtained by redispersing 0.02 ml of the slurry mixture obtained after wet grinding in 10 ml of distilled water, and 2) an another test solution obtained by redispersing 0.01 g of the dried powder was redispersed in 8 ml of distilled water, by using a laser scattering particle size analyzer (LA-910, Horiba). The redispersion was conducted by shaking the mixture with a hand.

As a result, as shown tables 5 and 6, the case of the amounts of the dispersion agent being same or less than that of the surface stabilizer, the particle size of the active ingredient was a little lager as compared to the case that the amounts of the dispersion agent was lager than the surface stabilizer. In addition, like Example 1, it makes no particle size different of the active ingredient between when the slurry mixture was redispersed in distilled water after wet grinding (table 5) and when the drying powder was redispersed (table 6).

TABLE 5

| | Dispersion | Surface | | | | |
|---|---|---|---|---|---|---|
| Active | agent | stabilizer | Particle size range | | | Average |
| ingredient | Lactose | SLS | D10 | D50 | D90 | particle size |
| Cilostazol | 360 g | 90 g | 140 | 180 | 250 | 190 |
| | 225 g | 225 g | 230 | 330 | 470 | 340 |
| | 90 g | 360 g | 230 | 340 | 470 | 340 |

TABLE 6

| | Dispersion | Surface | | | | |
|---|---|---|---|---|---|---|
| Active | agent | stabilizer | Particle size range | | | Average |
| ingredient | Lactose | SLS | D10 | D50 | D90 | particle size |
| Cilostazol | 360 g | 90 g | 150 | 200 | 270 | 200 |
| | 225 g | 225 g | 240 | 350 | 490 | 360 |
| | 90 g | 360 g | 230 | 340 | 390 | 350 |

### Example 6

### Particle Size Variation of the Active Ingredient with the Kind of the Active Ingredient

Powders were prepared using cilostazol (Dongwoo Co., Ltd.), fenofibrate (Sigma) or itraconazole (Pacificpharma Corporation) as an active ingredient, as follows:

1.2 g of each of the active ingredient, 0.2 g of hydroxypropyl cellulose and 1.2 g of sucrose were added to 6.1 g of distilled water containing a saturated solution of sucrose, an appropriate amount of zirconia bead having the average particle size of 1 mm were added thereto, and the mixture was roll ground at a rate of 108 rpm for 5 days. The slurry mixture thus obtained was screened to remove the beads, and the residue was high-pressure filtered (pressure: 640 mmHg) using the sound pressure, followed by vacuum drying to obtain powders.

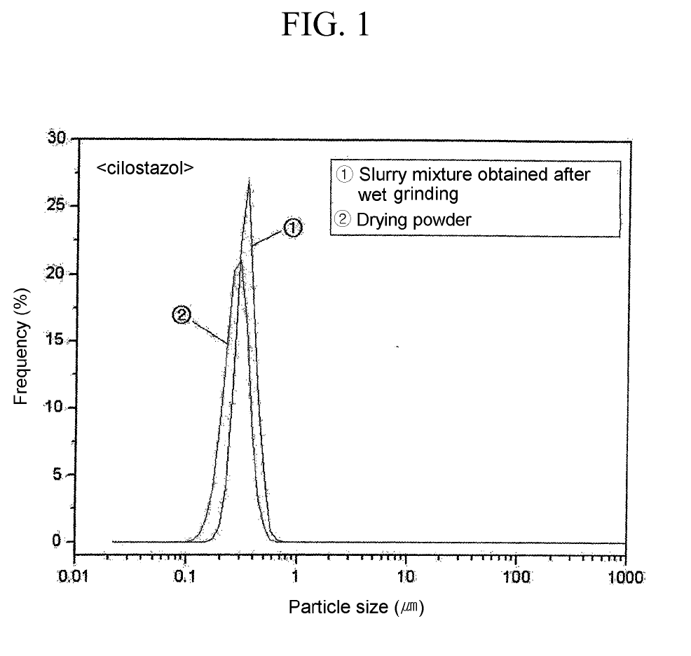

In the above process, the averaged particle size of the active ingredient was measured for 1) a test solution obtained by redespersing 0.2 ml of the slurry mixture obtained after wet grinding in 5 ml of distilled water, and 2) an another test solution obtained by redispersing 0.01 g of the dried powder in 5 ml of distilled water, by using a laser scattering particle size analyzer (LA-910, Horiba). As a result, as shown FIG. 1, it makes no particle size different of the active ingredient between when the slurry mixture was redispersed in the distilled water after wet grinding and when the drying powder was redispersed. The results of using fenofibrate or itraconazole as the active ingredient other than cilostazol representatively shown in FIG. 1 was also similar to above mentioned results.

Accordingly, the particle size of the active ingredient remained at nano-level when powders were redispersed in an aqueous solution. The results of the particle sizes of the active ingredient are shown in Table 7.

TABLE 7

| | | | | Average |
|---|---|---|---|---|
| Active | Surface | Dispersion | Range of | particle |
| ingredient | stabilizer | agent | particle size | size (nm) |
| Cilostazol | HPC | SUCROSE | 120~228 | 170 |
| Fenofibrate | | | 237~421 | 322 |
| Itraconazole | | | 62~234 | 132 |

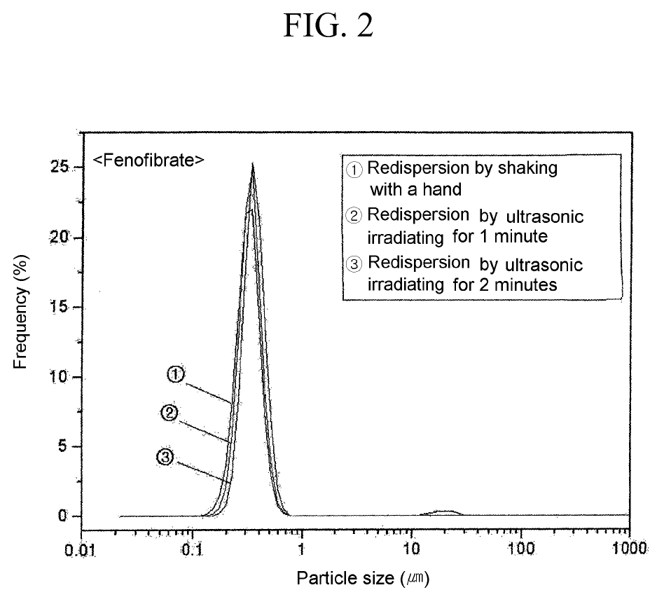

In order to observe the particle size variation of the active ingredient with the redispersion method, the average particle size of the active ingredient was measured for 1) a test solution which powders were redispersed after shaking with a hand, and 2) an another test solution obtained by redispersing in distilled water by the same method as mentioned above. The representative results of using fenofibrate are shown in FIG. 2.

As shown FIG. 2, the redispersion method did not significantly affected to the particle size of the active ingredient, and thus, the inventive samples have good redispersion properties.

### Test Example 4

### Bioavailability Test of the Powder Prepared According to the Present Invention

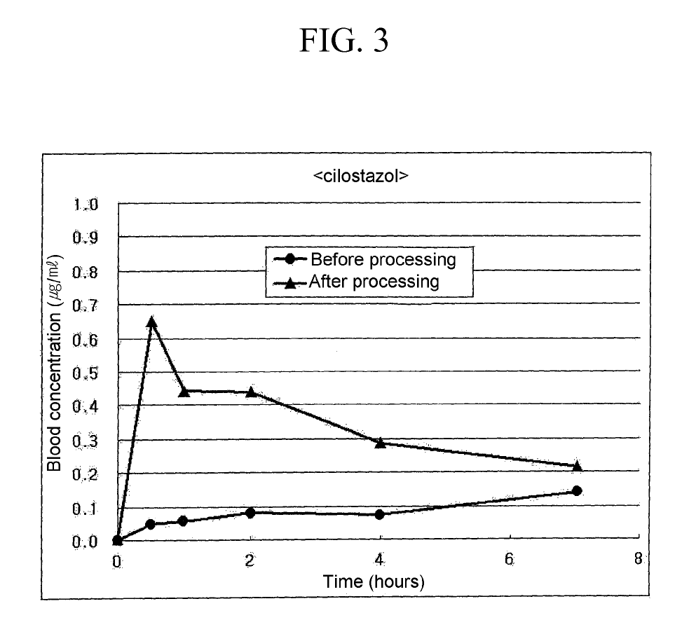

In order to investigate the bioavailability of the powder according to the present invention, cilostazol powder prepared in Example 6 and unprocessed cilostazole as a control group (average particle size: 3 to 5 μm, Dongwoo Co., Ltd.) were suspended in distilled water, respectively. The suspensions having equivalent amounts of cilostazole were each orally administered to three male rats fasted for 12 hours. Blood samples were taken from the ophthalmic vein of the rats immediately after the administration, and 0.5, 1, 2, 4 and 7 hours after the administration to determined the blood drug concentration changes with time. The results are shown in FIG. 3. Also the maximum blood concentration (Cmax, μg/ml) and the area under the blood drug concentration-time curve (AUC, μg*hr/ml) were calculated, and the results are shown in Table 8.

TABLE 8

| | Cilostazol powder | Cilostazol raw material |
|---|---|---|
| Cmax(μg/ml) | 0.65 ± 0.10 | 0.15 ± 0.08 |
| AUC(μg*hr/ml) | 2.36 ± 0.30 | 0.58 ± 0.09 |

As shown in FIG. 3 and Table 8, the maximum blood concentration (Cmax, μg/ml) and the area under the blood drug concentration-time curve for the cilostazole powder prepared according to the present invention become higher by approximately 4-fold, respectively, as compared to the results obtained for the unprocessed cilostazole. Therefore the inventive powders show markedly an enhanced bioavailability.

While the embodiments of the subject invention have been described and illustrated, it is obvious that various changes and modifications can be made therein without departing from the spirit of the present invention which should be limited only by the scope of the appended claims.

What is claimed is:

1. A method for preparing a powder composition comprising nanoparticles of a sparingly water-soluble drug comprising:
1) dispersing particles of the sparingly water-soluble drug, a surface stabilizer and a dispersion agent in a saturated aqueous solution of the dispersion agent to obtain a dispersion;
2) mixing and grinding the dispersion obtained in step 1) to obtain a homogenized dispersion; and
3) centrifuging or high-pressure filtering the homogenized dispersion obtained in step 2) to isolate a solid, and drying the solid to obtain a powder.

2. The method of claim 1, wherein the sparingly water-soluble drug is selected from the group consisting of non-steroidal anti-inflammatory drugs including acetaminophen, acetylsalicylic acid, ibuprofen, penbuprofen, fenoprofen, flubiprofen, indomethacin, naproxen, etorolac, ketoprofen, dexibuprofen, piroxicam and aceclofenac; immunosuppressants or therapeutic agents for atopic dermatitis including cyclosporine, tacrolimus, rapamycin, mycophenylate and pimecrolimus; calcium channel blockers including nifedipine, nimodipine, nitrendipine, nilvadipine, felodipine, amlodipine and isradipine; angiotensin II receptor antagonists including valsartan, eprosartan, irebesartan, candersartan, telmisartan, olmesartan and losartan; therapeutic agents for hyperlipidemia inhibiting cholesterol synthesis including atorvastatin, lovastatin, simvastatin, fluvastatin, rosuvastatin and pravastatin; therapeutic agents for hyperlipidemia promoting cholesterol metabolism and secretion including gemfibrozil, fenofibrate, etofibrate and bezafibrate; therapeutic agents for diabetes including pioglitazone, rosiglitazone and metformin; lipase inhibitors including orlistat; antifungal drugs including itraconazole, amphotericin B, terbinafine, nystatin, griseofulvin, fluconazole and ketoconazole; liver protectors including biphenyl dimethyl dicarboxylate, silymarin and ursodesoxycholic acid; therapeutic agents for digestive tract disease including sopharcone, omeprazole, pantoprazole, famotidine, itopride and mesalazine; platelet aggregation inhibitors including cilostazol and clopidogrel; therapeutic agents for osteoporosis including raloxifene; antiviral agents including acyclovir, famciclovir, lamivudine and oseltamivir; antibiotics including clarithromycin, ciprofloxacin and cefuroxime; antiasthmatic drugs and antihistamines including pranlukast, budesonide and fexofenadine; hormones including testosteron, prednisolone, estrogen, cortisone, hydrocortisone and dexamethasone; antitumor agents including paclitaxel, docetaxel, paclitaxel derivatives, doxorubicin, adriamycin, daunomycin, campothecin, etoposide, teniposide and busulfan; and salts, pharmaceutical derivatives, and a mixture thereof.

3. The method of claim 2, wherein the sparingly water-soluble drug is selected from the group consisting of naproxen, tacrolimus, valsartan, simvastatin, fenofibrate, itraconazole, biphenyl dimethyl dicarboxylate, silymarin, sopharcone, pantoprazole, cilostazol, and salts, pharmaceutical derivatives and a mixture thereof.

4. The method of claim 1, which further comprises the step of treating the sparingly water-soluble drug to form particles having an average particle size of less than 100 μm, before conducting step 1).

5. The method of claim 1, wherein the particles of sparingly water-soluble drug is contained in an amount ranging from 0.1 to 60% by weight in the saturated aqueous solution containing the dispersion agent.

6. The method of claim 1, wherein the surface stabilizer is selected from the group consisting of sodium dodecylsulfate, sodium dioctyl sulfosuccinate, lecithin, phospholipid, polyoxyethylene sorbitan fatty acid ester, potassium sorbate, poloxamer, prophylene glycol, methyl cellulose, ethyl cellulose, hydroxymethyl cellulose, hydroxyethyl cellulose, hydroxypropyl cellulose, hydroxypropyl methylcellulose, carboxymethyl cellulose, benzethonium chloride, benzalkonium chloride, sorbic acid, benzoic acid, sodium benzoate, propylparaben, methylparaben, polyvinylalcohol, polyvinylpyrrolidone, alginic acid, sodium alginate and a mixture thereof.

7. The method of claim 6, wherein the surface stabilizer is selected from the group consisting of hydroxypropyl cellulose, poloxamer, polyvinylpyrrolidone and a mixture thereof.

8. The method of claim 1, wherein the surface stabilizer is used in an amount ranging from 0.0001 to 90% by weight based on the weight of the sparingly water-soluble drug.

9. The method of claim 1, wherein the dispersion agent is selected from the group consisting of monosaccharides, disaccharides and trisaccharides including lactose, sucrose, raffinose, mannitol, trehalose, sorbitol, xylitol, glycerol, dextrose and fructose, and a mixture thereof.

10. The method of claim 1, wherein the dispersion agent is used in an amount ranging from 0.1 to 200% by weight based on the weight of the sparingly water-soluble drug.

11. The method of claim 1, wherein the mixing and grinding process in step 2) is conducted by wet grinding using a dispersion mill including a ball mill, an oscillating mill and a bead mill; ultrasonic irradiation; or hearing force grinding process.

12. The method of claim 1, wherein the homogenized dispersion obtained in step 2) has an apparent viscosity ranging from 1 to 100,000 centipoises.

13. The method of claim 1, wherein the centrifuging process in step 3) is conducted at a rate ranging from 500 to 200,000 rpm and a temperature ranging from 0 to 50° C.

14. The method of claim 1, wherein the high-pressure filtering process in step 3) is conducted at a pressure ranging from 200 to 2000 mmHg and a temperature ranging from 0 to 50° C.

15. The method of claim 1, wherein the drying process in step 3) is conducted by freeze drying or spray drying.

16. A powder composition comprising nanoparticles of a sparingly water-soluble drug prepared by the method according to claim 1, which shows a particle size distribution of 10 to 1000 nm for 10 to 90% of the drug particles determined based on a particle size normal distribution curve obtained for the powder and an average particle size of 10 to 400 nm in an aqueous medium.

17. A pharmaceutical composition comprising the powder composition of claim 16 together with a pharmaceutically acceptable carrier.

18. The pharmaceutical composition of claim 17, which is of a preparation form selected from the group consisting of granules, powders, syrups, liquids, suspensions, tablets, capsules, troches or pills for oral administration, and transdermal systems, lotions, ophthalmic ointments, ointments, plasters and pressure sensitive adhesives, cataplasmas, creams, pastes, suspensions, liquids, injections or suppositories for parenteral administration.
